(19) United States
(12) Patent Application Publication
(10) Pub. No.: US 2013/0137897 A1
(43) Pub. Date: May 30, 2013

(54) PROCESS FOR PREPARING SULFUR-CONTAINING 2-KETOCARBOXYLATE COMPOUND

(71) Applicants: Kazuyasu Tani, Osaka-shi (JP); Taro Hirose, Osaka-shi (JP); Koji Hagiya, Osaka-shi (JP)

(72) Inventors: Kazuyasu Tani, Osaka-shi (JP); Taro Hirose, Osaka-shi (JP); Koji Hagiya, Osaka-shi (JP)

(73) Assignee: SUMITOMO CHEMICAL COMPANY, LIMITED, Tokyo (JP)

(21) Appl. No.: 13/816,123

(22) PCT Filed: Aug. 26, 2011
(86) PCT No.: PCT/JP2011/069964

§ 371 Date: Feb. 8, 2013

(30) Foreign Application Priority Data
Aug. 27, 2010 (JP) 2010-190568

(51) Int. Cl. C07C 319/20

(52) U.S. Cl. CPC C07C 319/20
USPC 562/577

(57) ABSTRACT

The present invention provides a novel process for preparing a sulfur-containing 2-ketocarboxylate compound without using any enzyme. The process comprises a step of oxidizing a hydroxyacetate compound having an optionally-substituted sulfur-containing hydrocarbon group at 2-position in the presence of a vanadium compound. Preferably, the step is carried out further in the presence of oxygen, and more preferably, in the presence of an organic solvent.

### TECHNICAL FIELD

The present invention relates to a process for preparing a sulfur-containing 2-ketocarboxylate compound.

### BACKGROUND ART

It is known that sulfur-containing 2-ketocarboxylate compounds such as 4-methylthio-2-oxobutyric acid are, for example, useful intermediates for preparing medicaments and agrochemicals.

A conventional process for preparing a sulfur-containing 2-ketocarboxylate compound is disclosed in, for example, Non-patent Document 1. In particular, Table 4 thereof discloses a process of oxidizing D-2-hydroxy-4-(methylthio)butyric acid with an enzyme to prepare 4-(methylthio)-2-oxobutyric acid.

### PRIOR ART DOCUMENTS

Non-patent Document 1: Applied Microbiology and Biotechnology, 1988, vol. 28, p. 433-439

### SUMMARY OF INVENTION

### Technical Problem

In the above-mentioned process, it is necessary to use an enzyme as a reagent. On the other hand, the purpose of the present invention is to provide a novel process for preparing a sulfur-containing 2-ketocarboxylate compound without using any enzyme.

### Solution to Problem

The present inventors have extensively studied on the process of a sulfur-containing 2-ketocarboxylic acid, and then completed the present invention.

Namely, the present inventions are as follows:

[1] A process for preparing a sulfur-containing 2-ketocarboxylate compound comprising a step of oxidizing a hydroxyacetate compound having an optionally-substituted sulfur-containing hydrocarbon group at 2-position in the presence of a vanadium compound. The sulfur-containing hydrocarbon group used herein means a group comprising sulfur, carbon and hydrogen atoms.

[2] The process of [1] wherein the step of oxidizing a hydroxyacetate compound having an optionally-substituted sulfur-containing hydrocarbon group at 2-position is carried out in the presence of oxygen.

[3] The process of [1] or [2] wherein the step of oxidizing a hydroxyacetate compound having an optionally-substituted sulfur-containing hydrocarbon group at 2-position is carried out in the presence of an organic solvent.

[4] The process of [3] wherein the organic solvent is at least one solvent selected from the group consisting of a ketone solvent, a nitrile solvent and an aromatic solvent.

[5] The process of any one of [1] to [4] wherein the vanadium compound is at least one compound selected from the group consisting of a trivalent vanadium compound, a tetravalent vanadium compound and a pentavalent vanadium compound.

[6] The process of any one of [1] to [5] wherein the hydroxyacetate compound having an optionally-substituted sulfur-containing hydrocarbon group at 2-position is a compound of Formula (1):

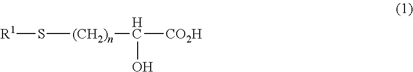

wherein R¹ is an optionally-substituted C₁₋₁₂ alkyl group, or an optionally-substituted C₃₋₁₂ cycloalkyl group; and n is an integer of 1 to 4, and

the sulfur-containing 2-ketocarboxylate compound is a compound of Formula (2):

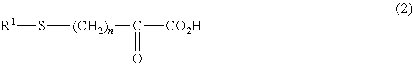

wherein R¹ and n are as defined above.

[7] The process of [6] wherein R¹ is methyl group and n is 2.

### Effects of the Invention

The present invention provides a novel process for preparing a sulfur-containing 2-ketocarboxylate compound without using any enzyme.

### DESCRIPTION OF EMBODIMENTS

Hereinafter, the present invention is explained in detail.

The process for preparing a sulfur-containing 2-ketocarboxylate compound of the present invention comprises a step of oxidizing a hydroxyacetate compound having an optionally-substituted sulfur-containing hydrocarbon group at 2-position. Hereinafter, a hydroxyacetate compound having an optionally-substituted sulfur-containing hydrocarbon group at 2-position is optionally referred to as a sulfur-containing hydroxyacetate compound. The oxidation of the sulfur-containing hydroxyacetate compound is optionally referred to as the present reaction. In the present reaction, —CH(OH)— in the sulfur-containing hydroxyacetate compound is oxidized to —CO—, and thereby the sulfur-containing hydroxyacetate compound is converted into the correspondent sulfur-containing 2-ketocarboxylate compound.

In the sulfur-containing hydroxyacetate compound, the sulfur-containing hydrocarbon group comprises sulfur, carbon and hydrogen atoms, and the hydrogen atom included in the sulfur-containing hydrocarbon group may be substituted with any group which is inert in the present reaction.

The sulfur-containing hydrocarbon group may be a saturated sulfur-containing hydrocarbon group without any double/multiple bond or an unsaturated sulfur-containing hydrocarbon group with one or more double bonds.

The saturated sulfur-containing hydrocarbon group may be in a straight or branched chain form or in a cyclic form. Hereinafter, the straight or branched saturated sulfur-containing hydrocarbon group is optionally referred to as a sulfur-containing saturated open-chain hydrocarbon group, and the cyclic saturated sulfur-containing hydrocarbon group is optionally referred to as a sulfur-containing saturated cyclic hydrocarbon group.

The sulfur-containing saturated open-chain hydrocarbon group includes, for example, methylthiomethyl group, ethylthiomethyl group, propylthiomethyl group, isopropylthiomethyl group, tert-butylthiomethyl group, 1-(methylthio)ethyl group, 2-(methylthio)ethyl group, 1-(ethylthio)ethyl group, 2-(ethylthio)ethyl group, 1-(propylthio)ethyl group, 2-(propylthio)ethyl group, 2-(isopropylthio)ethyl group, 2-(tert-butylthio)ethyl group, 1-(methylthio)propyl group, 2-(methylthio)propyl group, 3-(methylthio)propyl group, 3-(ethylthio)propyl group, 3-(propylthio)propyl group, 3-(isopropylthio)propyl group and 2,3-(dimethylthio)propyl group.

The sulfur-containing saturated cyclic hydrocarbon group includes, for example, cyclopropylthiomethyl group, cyclobutylthiomethyl group, cyclopentylthiomethyl group, cyclohexylthiomethyl group, 2-(methylthio)cyclopropyl group, 2-(methylthio)cyclobutyl group, 2-(methylthio)cyclopentyl group, 2-(methylthio)cyclohexyl group, 4-(methylthio)cyclohexyl group, 2-methyl-4-(methylthio)cyclohexyl group, 2,4-(dimethylthio)cyclohexyl group, 2-thiacyclohexyl group and 4-thiacyclohexyl group.

The sulfur-containing unsaturated hydrocarbon group includes, for example, vinylthiomethyl group, 1-(vinylthio)ethyl group, 2-(vinylthio)ethyl group, 4-methylthio-1-butenyl group, 4-methylthio-2-butenyl group, 2-methylthiophenyl group, 3-methylthiophenyl group, 4-methylthiophenyl group, 2-methyl-4-methylthiophenyl group, 2,4-(dimethylthio)phenyl group, phenylthiomethyl group, 1-(phenylthio)ethyl group, 2-(phenylthio)ethyl group, benzylthiomethyl group, 1-(benzylthio)ethyl group, 2-(benzylthio)ethyl group, 2-thienyl group, 3-thienyl group and 2-methyl-3-thienyl group.

Examples of the inert substituent which does not interfere with the present reaction include at least one substituent selected from the following Group P1:

an alkyloxy group having 1 to 12 carbon atoms such as methoxy group, ethoxy group, propyloxy group, isopropyloxy group, butyloxy group, isobutyloxy group, sec-butyloxy group, tert-butyloxy group, pentyloxy group and hexyloxy group;

an aralkyloxy group having 7 to 12 carbon atoms such as benzyloxy group;

a cycloalkyloxy group having 3 to 8 carbon atoms such as cyclopropyloxy group, cyclobutyloxy group, cyclopentyloxy group and cyclohexyloxy group;

an aryloxy group having 6 to 12 carbon atoms such as phenoxy group, 2-methylphenoxy group, 4-methylphenoxy group and 4-phenylphenyl group;

a perfluoroalkyloxy group having 1 to 6 carbon atoms such as trifluoromethoxy group and pentafluoroethoxy group;

an optionally-substituted amino group (wherein the substituted amino group has, for example, 1 to 12 carbon atoms) such as amino group, methylamino group, dimethylamino group, benzylamino group, tert-butoxycarbonylamino group and benzyloxycarbonylamino group;

an acyl group having 2 to 12 carbon atoms such as acetyl group, propionyl group, butyryl group, isobutyryl group, valeryl group, isovaleryl group, pivaloyl group and benzoyl group;

an acyloxy group having 2 to 12 carbon atoms such as acetyloxy group, propionyloxy group, butyryloxy group, isobutyryloxy group, valeryloxy group, isovaleryloxy group, pivaloyloxy group and benzoyloxy group; and

a halogen atom such as fluorine atom and chlorine atom.

Among the above-defined Group P1, the alkyloxy (alkoxy) group having 1 to 12 carbon atoms and the aryloxy group having 6 to 12 carbon atoms may be further substituted with, for example, at least one substituent selected from the following Group P2:

an alkyloxy group having 1 to 12 carbon atoms such as methoxy group, ethoxy group, propyloxy group, isopropyloxy group, butyloxy group, isobutyloxy group, sec-butyloxy group, tert-butyloxy group, pentyloxy group and hexyloxy group;

an aryloxy group having 6 to 12 carbon atoms such as phenoxy group, 2-methylphenoxy group, 4-methylphenoxy group and 4-phenylphenyl group; and

a halogen atom such as fluorine atom and chlorine atom.

The optionally-substituted sulfur-containing hydrocarbon group is preferably an optionally-substituted sulfur-containing saturated hydrocarbon group; more preferably an optionally-substituted sulfur-containing saturated open-chain hydrocarbon group; and even more preferably a group represented by R¹—S—(CH₂)ₙ— wherein R¹ is an optionally-substituted C₁₋₁₂ alkyl group or an optionally-substituted C₃₋₁₂ cycloalkyl group, and n is an integer of 1 to 4.

In the above-defined R¹—S—(CH₂)ₙ—, the C₁₋₁₂ alkyl group indicated in the definition of R¹ includes, for example, methyl group, ethyl group, propyl group, isopropyl group, butyl group, isobutyl group, tert-butyl group and octyl group. The C₃₋₁₂ cycloalkyl group indicated in the definition of R¹ includes, for example, cyclopropyl group, cyclobutyl group, cyclopentyl group and cyclohexyl group.

The substitutent used in the definition of R¹, i.e. in “the optionally-substituted C₁₋₁₂ alkyl group and the optionally-substituted C₃₋₁₂ cycloalkyl group” includes, for example, an inert substituent which does not interfere with the present reaction. Preferably, the inert substituent may be at least one selected from the following Group P3: an aryl group having 6 to 20 carbon atoms such as phenyl group, 1-naphthyl group, 2-naphthyl group and 4-methylphenyl group;

an alkyloxy group having 1 to 12 carbon atoms such as methoxy group, ethoxy group, propyloxy group, isopropyloxy group, butyloxy group, isobutyloxy group, sec-butyloxy group, tert-butyloxy group, pentyloxy group and hexyloxy group;

an aralkyloxy group having 7 to 12 carbon atoms such as benzyloxy group;

a cycloalkyloxy group having 3 to 8 carbon atoms such as cyclopropyloxy group, cyclobutyloxy group, cyclopentyloxy group and cyclohexyloxy group;

an aryloxy group having 6 to 12 carbon atoms such as phenoxy group, 2-methylphenoxy group, 4-methylphenoxy group and 4-phenylphenoxy group;

a perfluoroalkyloxy group having 1 to 6 carbon atoms such as trifluoromethoxy group and pentafluoroethoxy group;

an optionally-substituted amino group (wherein the substituted amino group has, for example, 1 to 12 carbon atoms) such as amino group, methylamino group, dimethylamino group, benzylamino group, tert-butoxycarbonylamino group and benzyloxycarbonylamino group;

an acyl group having 2 to 12 carbon atoms such as acetyl group, propionyl group, butyryl group, isobutyryl group, valeryl group, isovaleryl group, pivaloyl group and benzoyl group;

an acyloxy group having 2 to 12 carbon atoms such as acetyloxy group, propionyloxy group, butyryloxy group, isobutyryloxy group, valeryloxy group, isovaleryloxy group, pivaloyloxy group and benzoyloxy group; and

a halogen atom such as fluorine atom and chlorine atom.

Among the above-defined Group P3, the aryl group having 6 to 20 carbon atoms, the alkyloxy (alkoxy) group having 1 to 12 carbon atoms and the aryloxy group having 6 to 12 carbon atoms may be substituted with, for example, at least one substituent selected from the above-defined Group P2.

The substituted C₁₋₁₂ alkyl group and the substituted C₃₋₁₂ cycloalkyl group in R¹ includes, for example, benzyl group, naphthalene-1-ylmethyl group, naphthalene-2-ylmethyl group, 4-methylbenzyl group, 3,4-dimethylbenzyl group, 4-methoxybenzyl group, 3,4-dimethoxybenzyl group, 4-phenylbenzyl group, 4-phenoxybenzyl group, methoxymethyl group, ethoxymethyl group, isopropyloxymethyl group, butyloxymethyl group, isobutyloxymethyl group, sec-butyloxymethyl group, tert-butyloxymethyl, phenoxymethyl group, 2-methylphenoxymethyl group, 4-methylphenoxymethyl group, 1-phenylethyl group, 2-phenylethyl group, 1-(naphthalene-1-yl)ethyl group, 1-(naphthalene-2-yl)ethyl group, 1-(4-methylphenyl)ethyl group, 1-(3,4-dimethylphenyl)ethyl group, 1-(4-methoxyphenyl)ethyl group, 1-(3,4-dimethoxyphenyl)ethyl group, 1-(4-phenylphenyl)ethyl group, 1-(4-phenoxyphenyl)ethyl group, 2-(methoxy)ethyl group, 2-(ethoxy)ethyl group, 2-(isopropyloxy)ethyl group, 2-(butyloxy)ethyl group, 2-(isobutyloxy)ethyl group, 2-(sec-butyloxy)ethyl group, 2-(tert-butyloxy)ethyl group, 2-(phenoxy)ethyl group, 2-(2-methylphenoxy)ethyl group, 2-(4-methylphenoxy)ethyl group, 2-phenylcyclopropyl group and 4-phenylcyclohexyl group.

R¹ is preferably an optionally-substituted C₁₋₁₂ alkyl group; more preferably an alkyl group having 1 to 4 carbon atoms which may be optionally substituted with phenyl group; and even more preferably methyl group.

When the optionally-substituted sulfur-containing hydrocarbon group at 2-position of the hydroxyacetate compound is represented by R¹—S—(CH₂)ₙ—, the hydroxyacetate compound is depicted as a compound of Formula (1):

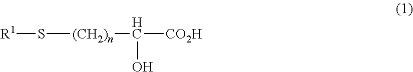

wherein R¹ and n are as defined above. Hereinafter, the compound of Formula (1) is optionally referred to as Compound (1). A preferred example of the sulfur-containing hydroxyacetate compound is Compound (1).

Compound (1) includes, for example, 2-hydroxy-3-methylthiopropionic acid, 3-tert-butylthio-2-hydroxypropionic acid, 3-benzylthio-2-hydroxypropionic acid, 3-ethylthio-2-hydroxypropionic acid, 2-hydroxy-4-(methylthio)butyric acid, 4-ethylthio-2-hydroxybutyric acid, 2-hydroxy-4-propylthiobutyric acid, 4-benzylthio-2-hydroxybutyric acid, 2-hydroxy-5-(methylthio)pentanoic acid, 5-(ethylthio)-2-hydroxypentanoic acid, 2-hydroxy-5-(propylthio)pentanoic acid, 5-(benzylthio)-2-hydroxypentanoic acid, 2-hydroxy-6-(methylthio)hexanoic acid, 6-(ethylthio)-2-hydroxyhexanoic acid, 2-hydroxy-6-(propylthio)hexanoic acid, 6-(benzylthio)-2-hydroxyhexanoic acid and salts thereof.

Compound (1) may be commercially available or may be prepared according to the process described in, for example, JP-2006-109834 A.

Hereinafter, the step of oxidizing the sulfur-containing hydroxyacetate compound is explained. In the present reaction, the sulfur-containing hydroxyacetate compound is converted into the correspondent sulfur-containing 2-ketocarboxylate compound.

The present reaction is carried out in the presence of a vanadium compound.

The vanadium compound includes, for example, a divalent vanadium compound, a trivalent vanadium compound, a tetravalent vanadium compound and a pentavalent vanadium compound. The divalent vanadium compound means that the compound comprises a vanadium atom having a valence of two, the trivalent vanadium compound means that the compound comprises a vanadium atom having a valence of three, the tetravalent vanadium compound means that the compound comprises a vanadium atom having a valence of four, and the pentavalent vanadium compound means that the compound comprises a vanadium atom having a valence of five.

The divalent vanadium compound includes, for example, vanadium oxide (VO).

The trivalent vanadium compound includes, for example, vanadium (III) having acetylacetonates as a ligand [e.g. vanadium (III) tris(acetylacetonate)].

The tetravalent vanadium compound includes, for example, tetravalent oxyvanadium compounds having acetyl-acetonates as a ligand [e.g. vanadium (IV) oxyacetylacetonate], oxovanadium (IV) oxalate, and [2,2′-[1,2-ethanediyl bis[nitrilo-κN]methylidyne]]bis[phenolate-κO]](2-)]oxovanadium (IV).

The pentavalent vanadium compound includes, for example, vanadate compounds (V) such as sodium vanadate, potassium vanadate and ammonium vanadate; metavanadate compounds (V) such as sodium metavanadate, potassium metavanadate, and ammonium metavanadate; trialkoxy-oxovanadium (V); and oxo(2-propanolato)[2,6-pyridine-dicarboxylato(2-)-N1,O2,O6]vanadium (V).

Preferably, the vanadium compound is at least one compound selected from the group consisting of trivalent vanadium compounds, tetravalent vanadium compounds and pentavalent vanadium compounds. More preferably, the vanadium compound is at least one compound selected from the group consisting of vanadium (III) tris(acetylacetonate), oxovanadium (IV) oxalate, vanadium (IV) oxyacetylacetonate, trialkoxyoxovanadium (V), sodium metavanadate (V), potassium metavanadate (V), ammonium metavanadate (V), [2,2′-[1,2-ethanediyl bis[nitrilo-κN]methylidyne]]bis[phenolate-κO]](2-)]oxovanadium (IV), and oxo(2-propanolato)[2,6-pyridinedicarboxylato(2-)-N1,O2,O6]vanadium (V). Even more preferably, the vanadium compound is at least one compound selected from the group consisting of [2,2′-[1,2-ethanediyl bis[nitrilo-κN]methylidyne]]bis[phenolate-κO]](2-)]oxovanadium (IV), and oxo(2-propanolato)[2,6-pyridinedicarboxylato(2-)—N1,O2,O6]vanadium (V).

The amount of the vanadium compound used herein may vary depending on, for example, the concentration of the reaction mixture. The amount is preferably 0.001 mol or more per 1 mol of the sulfur-containing hydroxyacetate compound; and from a practical viewpoint, it should be 0.5 mol or less per 1 mol thereof.

Preferably, the present reaction is carried out in the presence of oxygen.

The oxygen used herein may be oxygen gas which is undiluted or diluted with an inert gas such as nitrogen, or oxygen from the air. In addition, the oxygen from the air may be diluted with an inert gas such as nitrogen.

The amount of oxygen used herein is preferably 1 mol or more per 1 mol of the sulfur-containing hydroxyacetate compound, and there is no upper limit thereof.

Preferably, the present reaction is carried out in the presence of an organic solvent.

The organic solvent is at least one solvent selected from the group consisting of, for example, a ketone solvent, a nitrile solvent and an aromatic solvent. The ketone solvent includes, for example, acetone, methylethylketone and methylisobutylketone. The nitrile solvent includes, for example, acetonitrile and propionitrile. The aromatic solvent includes, for example, toluene, xylene, ethylbenzene and benzotrifluoride. Preferably, the organic solvent is at least one solvent selected from the group consisting of acetone, acetonitrile and benzotrifluoride.

The amount of the solvent used herein is not limited to a particular amount, but from the viewpoint of volumetric efficiency, the amount is preferably not more than 100 parts by weight per 1 part by weight of the sulfur-containing hydroxyacetate compound.

In the present reaction, the addition order of each reagent is not limited. A preferred embodiment of the procedure includes, for example, a procedure which comprises mixing the sulfur-containing hydroxyacetate compound, an organic solvent and a vanadium compound, and then mixing the mixture and oxygen.

The present reaction may be carried out under a reduced pressure condition, a normal pressure condition or a pressurized condition. Preferably, it is carried out under a normal pressure condition or a pressurized condition; and more preferably, it is carried out under a normal pressure condition. The pressurized condition is, for example, 0.2 to 10 MPaG (gauge pressure).

The reaction temperature of the present reaction may vary depending on factors such as the concentration of the sulfur-containing hydroxyacetate compound contained in the reaction mixture and the amount of the vanadium compound. The reaction temperature is in the range of, preferably 0° C. to 130° C., more preferably 10° C. to 100° C., further more preferably 20° C. to 80° C., and even more preferably room temperature (about 25° C.) to 55° C. When the reaction temperature is lower than 0° C., the present reaction tends to proceed slowly, while when it is higher than 130° C., the selectivity rate of the present invention tends to decrease.

The reaction can be monitored by analytical methods such as gas chromatography, high performance liquid chromatography, thin-layer chromatography, nuclear magnetic resonance spectrum analysis, and infrared absorption spectrum analysis.

For example, the sulfur-containing 2-ketocarboxylate compound can be isolated as follows. After the reaction is completed, the resultant reaction mixture is concentrated, and the mixture is optionally neutralized with mineral acids such as sulfuric acid and hydrochloric acid. Then, the mixture can be concentrated, cooled, or mixed with, for example, acetone to isolate the sulfur-containing 2-ketocarboxylate compound as a solid. In case that the sulfur-containing 2-ketocarboxylate compound is a lipophilic compound, it may be isolated as follows. After the reaction is completed, the resultant reaction mixture is mixed with a water-immiscible solvent, extracted, concentrated, and cooled to isolate the sulfur-containing 2-ketocarboxylate compound. The water-immiscible solvent includes, for example, an ester solvent such as ethyl acetate and an ether solvent such as methyl tert-butyl ether. In addition, the amount of the water-immiscible solvent used herein is not limited to a particular amount.

The isolated sulfur-containing 2-ketocarboxylate compound can be purified by methods such as distillation, column chromatography and crystallization.

The sulfur-containing 2-ketocarboxylate compound prepared by the above-mentioned process has an optionally-substituted sulfur-containing hydrocarbon group. The optionally-substituted sulfur-containing hydrocarbon group in the sulfur-containing 2-ketocarboxylate compound is the same as that of the above-mentioned sulfur-containing hydroxyacetate compound.

The sulfur-containing 2-ketocarboxylate compound includes, for example, 3-methylthio-2-oxopropionic acid, 3-tert-butylthio-2-oxopropionic acid, 3-benzylthio-2-oxopropionic acid, 3-ethylthio-2-oxopropionic acid, 4-methylthio-2-oxobutyric acid, 4-ethylthio-2-oxobutyric acid, 2-oxo-4-propylthiobutyric acid, 4-benzylthio-2-oxobutyric acid, 5-methylthio-2-oxopentanoic acid, 5-(ethylthio)-2-oxopentanoic acid, 2-oxo-5-(propylthio)pentanoic acid, 5-(benzylthio)-2-oxopentanoic acid, 6-methylthio-2-oxohexanoic acid, 6-(ethylthio)-2-oxohexanoic acid, 2-oxo-6-(propylthio)hexanoic acid, 6-(benzylthio)-2-oxohexanoic acid and salts thereof.

### Example

Hereinafter, the present invention is illustrated in more detail with some examples.

In the following examples, each reaction mixture was analyzed with high performance liquid chromatograph (manufactured by Shimadzu Corporation) under the analysis conditions shown below, and each conversion rate and each selectivity rate were calculated using the formulae shown below.

### Analysis Conditions:

LC column: Lichrosorb-RP-8

Column temperature: 40° C.

Mobile phase: acetonitrile/water=5/95
Additive agent: sodium 1-pentanesulfonate
Concentration of additive agent: 2.5 mmol/L
pH of mobile phase: pH 3 (adjusted by adding 40% phosphoric acid)

Flow rate: 1.5 mL/min

Detection wavelength: 210 nm

Measurement time: 60 min

Calculation of Conversion rate:

Conversion rate (%)=100(%)−[Peak area of Compound (1)(%)]

Calculation of Selectivity rate:

Selectivity rate (%)=[Peak area of Compound (2)]/(Peak area of all products)×100

### Example 1

A 25 mL reactor equipped with a magnetic stirring bar was charged with 150 mg of 2-hydroxy-4-(methylthio)butyric acid, 5 g of acetone and 12 mg of triisopropoxyoxovanadium (V), and the mixture was stirred for 38 hours at room temperature under oxygen atmosphere. After the reaction was completed, the solvent was distilled away from the reaction mixture, and to the resultant mixture was added 1 g of 1 N hydrochloric acid. A portion of the reaction mixture was analyzed by high performance liquid chromatography to find that the conversion rate of 2-hydroxy-4-(methylthio)butyric acid was 16% and the selectivity rate of 4-(methylthio)-2-oxobutyric acid was 51%.

### Example 2

A 25 mL reactor equipped with a magnetic stirring bar was charged with 150 mg of 2-hydroxy-4-(methylthio)butyric acid, 5 g of acetone and 13 mg of vanadium (IV) oxyacetylacetonate, and the mixture was stirred for 6 hours at room temperature under oxygen atmosphere. After the reaction was completed, the solvent was distilled away from the reaction mixture, and to the resultant mixture was added 1 g of 1 N hydrochloric acid. A portion of the reaction mixture was analyzed by high performance liquid chromatography to find that the conversion rate of 2-hydroxy-4-(methylthio)butyric acid was 11% and the selectivity rate of 4-(methylthio)-2-oxobutyric acid was 54%.

### Example 3

A 25 mL reactor equipped with a magnetic stirring bar was charged with 150 mg of 2-hydroxy-4-(methylthio)butyric acid, 5 g of acetonitrile and 13 mg of vanadium (IV) oxyacetylacetonate, and the mixture was stirred for 19 hours at room temperature under oxygen atmosphere. After the reaction was completed, the solvent was distilled away from the reaction mixture, and to the resultant mixture was added 1 g of 1 N hydrochloric acid. A portion of the reaction mixture was analyzed by high performance liquid chromatography to find that the conversion rate of 2-hydroxy-4-(methylthio)butyric acid was 9% and the selectivity rate of 4-(methylthio)-2-oxobutyric acid was 55%.

### Example 4

A 25 mL reactor equipped with a magnetic stirring bar was charged with 150 mg of 2-hydroxy-4-(methylthio)butyric acid, 1.5 g of benzotrifluoride, 0.75 g of acetonitrile, 0.75 g of acetone and 13 mg of vanadium (IV) oxyacetylacetonate, and the mixture was stirred for 24 hours at room temperature under oxygen atmosphere. After the reaction was completed, the solvent was distilled away from the reaction mixture, and to the resultant mixture was added 1 g of 1 N hydrochloric acid. A portion of the reaction mixture was analyzed by high performance liquid chromatography to find that the conversion rate of 2-hydroxy-4-(methylthio)butyric acid was 11% and the selectivity rate of 4-(methylthio)-2-oxobutyric acid was 41%.

### Example 5

A 25 mL reactor equipped with a magnetic stirring bar was charged with 150 mg of 2-hydroxy-4-(methylthio)butyric acid, 7.5 g of acetonitrile and 17 mg of vanadium (III) tris(acetylacetonate), and the mixture was stirred for 24 hours at room temperature under oxygen atmosphere. After the reaction was completed, the solvent was distilled away from the reaction mixture, and to the resultant mixture was added 1 g of 1 N hydrochloric acid. A portion of the reaction mixture was analyzed by high performance liquid chromatography to find that the conversion rate of 2-hydroxy-4-(methylthio)butyric acid was 17% and the selectivity rate of 4-(methylthio)-2-oxobutyric acid was 33%.

### Example 6

A 10 mL reactor equipped with a magnetic stirring bar was charged with 150 mg of 2-hydroxy-4-(methylthio)butyric acid, 3.75 g of acetone and 6 mg of ammonium metavanadate (V), and the mixture was stirred for 96 hours at room temperature under oxygen atmosphere. After the reaction was completed, the solvent was distilled away from the reaction mixture, and to the resultant mixture was added 1 g of 1 N hydrochloric acid. A portion of the reaction mixture was analyzed by high performance liquid chromatography to find that the conversion rate of 2-hydroxy-4-(methylthio)butyric acid was 7% and the selectivity rate of 4-(methylthio)-2-oxobutyric acid was 31%.

### Example 7

A 10 mL reactor equipped with a magnetic stirring bar was charged with 150 mg of 2-hydroxy-4-(methylthio)butyric acid, 7.5 g of acetonitrile and 8 mg of oxovanadium (IV) oxalate, and the mixture was stirred for 96 hours at room temperature under oxygen atmosphere. After the reaction was completed, the solvent was distilled away from the reaction mixture, and to the resultant mixture was added 1 g of 1 N hydrochloric acid. A portion of the reaction mixture was analyzed by high performance liquid chromatography to find that the conversion rate of 2-hydroxy-4-(methylthio)butyric acid was 5% and the selectivity rate of 4-(methylthio)-2-oxobutyric acid was 30%.

### Example 8

A 60 mL autoclave was charged with 1.50 g of 2-hydroxy-4-(methylthio)butyric acid, 7.57 g of acetonitrile, 1.02 g of triethylamine and 29 mg of a vanadium compound shown in the below Formula (C-1), i.e., oxo(2-propanolato)[2,6-pyridinedicarboxylato(2-)-N1,O2,O6]vanadium (V) which was prepared according to the process disclosed in Inorg. Chem., Vol. 35, p. 547-548 (1996), and the mixture was stirred. After the autoclave was pressurized to 0.8 MPaG (gauge pressure) with 30% oxygen/70% nitrogen, the mixture was heated to 50° C. and stirred for 24 hours. A portion of the reaction mixture was analyzed by high performance liquid chromatography to find that the conversion rate of 2-hydroxy-4-(methylthio)butyric acid was 87% and the selectivity rate of 4-(methylthio)-2-oxobutyric acid was 46%.

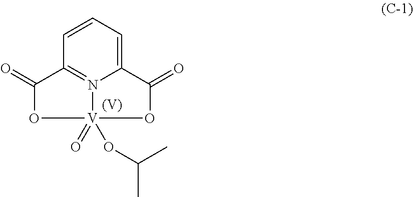

### Example 9

A 60 mL autoclave was charged with 1.50 g of 2-hydroxy-4-(methylthio)butyric acid, 6.40 g of acetonitrile, 0.89 g of triethylamine and 30 mg of a vanadium compound shown in the below Formula (C-2), i.e., [2,2′-[1,2-ethanediyl bis[nitrilo-κN]methylidyne]]bis[phenolate-κO]](2-)]oxovanadium (IV) which was prepared according to the process disclosed in Catal. Commun., Vol. 8, p. 1336-1340 (2007), and the mixture was stirred. After the autoclave was pressurized to 0.5 MPaG (gauge pressure), the mixture was heated to 50° C. and stirred for 18 hours. A portion of the reaction mixture was analyzed by high performance liquid chromatography to find that the conversion rate of 2-hydroxy-4-(methylthio)butyric acid was 86% and the selectivity rate of 4-(methylthio)-2-oxobutyric acid was 48%.

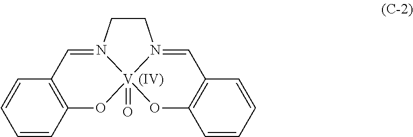

### INDUSTRIAL APPLICABILITY

It is known that sulfur-containing 2-ketocarboxylate compounds such as 4-methylthio-2-oxobutyric acid are, for example, useful intermediates for preparing medicaments and agrochemicals. The present invention is an industrially applicable process for preparing sulfur-containing 2-ketocarboxylate compounds.

1. A process for preparing a sulfur-containing 2-ketocarboxylate compound comprising a step of oxidizing a hydroxyacetate compound having an optionally-substituted sulfur-containing hydrocarbon group at 2-position in the presence of a vanadium compound.

2. The process of claim 1 wherein the step of oxidizing a hydroxyacetate compound having an optionally-substituted sulfur-containing hydrocarbon group at 2-position is carried out in the presence of oxygen.

3. The process of claim 1 wherein the step of oxidizing a hydroxyacetate compound having an optionally-substituted sulfur-containing hydrocarbon group at 2-position is carried out in the presence of an organic solvent.

4. The process of claim 3 wherein the organic solvent is at least one solvent selected from the group consisting of a ketone solvent, a nitrile solvent and an aromatic solvent.

5. The process of claim 1 wherein the vanadium compound is at least one compound selected from the group consisting of a trivalent vanadium compound, a tetravalent vanadium compound and a pentavalent vanadium compound.

6. The process of claim 1 wherein the hydroxyacetate compound having an optionally-substituted sulfur-containing hydrocarbon group at 2-position is a compound of Formula (1): 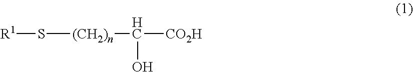
wherein R¹ is an optionally-substituted C₁₋₁₂ alkyl group, or an optionally-substituted C₃₋₁₂ cycloalkyl group; and n is an integer of 1 to 4, and
the sulfur-containing 2-ketocarboxylate compound is a compound of Formula (2): 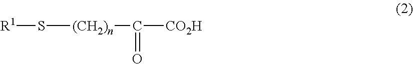
wherein R¹ and n are as defined above.

7. The process of claim 6 wherein R¹ is methyl group and n is 2.
